(19) United States
(12) Patent Application Publication
(10) Pub. No.: US 2025/0153149 A1
(43) Pub. Date: May 15, 2025

(54) INTEGRATED DIRECT AIR CAPTURE AND CONVERSION TO HYDROCARBONS

(71) Applicant: Battelle Memorial Institute, Richland, WA (US)

(72) Inventors: Jotheeswari Kothandaraman, Richland, WA (US); Robert A. Dagle, Richland, WA (US); Johnny Saavedra Lopez, Richland, WA (US); Shazia S. Satter, Richland, WA (US)

(21) Appl. No.: 18/941,842

(22) Filed: Nov. 8, 2024

Related U.S. Application Data
(60) Provisional application No. 63/547,942, filed on Nov. 9, 2023.

(51) Int. Cl. B01J 23/745; B01D 53/02; B01D 53/86; B01J 20/04; C07C 1/12

(52) U.S. Cl. CPC B01J 23/745; B01D 53/02; B01D 53/8671; B01J 20/043; C07C 1/12; B01D 2253/112; B01D 2253/25; B01D 2255/20738; B01D 2257/504; B01D 2258/06; C07C 2523/78

(57) ABSTRACT

A multifunctional material may include a solid inorganic sorbent. The multifunctional material may include a metal catalyst component, wherein the solid inorganic sorbent and metal catalyst component are integrated into a single material.

### CROSS-REFERENCE TO RELATED APPLICATION

This application claims the benefit of priority to U.S. Provisional Patent Application Ser. No. 63/547,942 entitled “INTEGRATED DIRECT AIR CAPTURE AND CONVERSION TO HYDROCARBONS,” filed Nov. 9, 2023, the disclosure of which is incorporated herein in its entirety by reference.

### STATEMENT AS TO RIGHTS TO DISCLOSURES MADE UNDER FEDERALLY-SPONSORED RESEARCH AND DEVELOPMENT

This invention was made with Government support under Contract DE-AC0576RL01830 awarded by the U.S. Department of Energy. The Government has certain rights in the invention.

### BACKGROUND

Direct air capture technology is a form of carbon dioxide (CO₂) removal that takes CO₂ from ambient, or still, air. The separated CO₂ can then be permanently stored deep underground, or it can be converted into products.

### SUMMARY

In some aspects, the techniques described herein relate to a multifunctional material for integrated capture and catalytic conversion of CO2, including: a solid inorganic sorbent; and a metal catalyst component, wherein the solid inorganic sorbent and metal catalyst component are integrated into a single material.

In some aspects, the techniques described herein relate to a method for integrated direct air capture and catalytic conversion of CO₂ to (C1-C10)hydrocarbyl products, including: contacting a multifunctional material including a solid inorganic sorbent and a metal catalyst component with air to capture CO₂; and hydrogenating the captured CO₂ to produce (C1-C10)hydrocarbyls.

### BRIEF DESCRIPTION OF THE FIGURES

The drawings illustrate generally, by way of example, but not by way of limitation, various aspects of the present disclosure.

FIG. 1A is a graph showing the CO₂ capture profile over K₂CO₃/Fe/C.

FIG. 1B is a graph showing the hydrogenation profile of the captured CO₂ (at heating rate of 20° C./min)

FIG. 1C is a graph showing the CO₂ conversion and selectivity of products formed in Region 1 at 320° C.,

FIG. 1D is a graph showing the comparison of the CO₂ conversion and selectivity of the products formed in Region 2 and 360° C.

FIG. 2A is a graph showing the comparison of the CO₂ adsorption capacity of K₂CO₃ on various materials pretreated at high temperature under H₂ flow.

FIG. 2B is a graph showing hydrogenation of the captured CO₂ over K₂CO₃/Fe/C/Al₂O₃.

FIG. 3A is a graph showing the nitrogen adsorption isotherm of K₂CO₃/Al₂O₃ (also referred as KA), K₂CO₃/Fe/C/Al₂O₃, and Fe/C.

FIG. 3B is a graph showing the pore size distribution Barrett-Joyner-Halenda (BJH) curves for K₂CO₃/Al₂O₃, and K₂CO₃/Fe/C/Al₂O₃.

FIG. 4A is a graph showing CO₂ capture and hydrogenation of captured CO₂ using Fe/KA.

FIG. 4B is a graph showing comparison of the N₂ adsorption isotherm of K₂CO₃/Al₂O₃, Fe/KA-Fresh, and Fe/KA-Spent (after hydrogenation).

FIG. 4C is a graph showing powder XRD of freshly captured CO₂ and spent Fe/KA.

FIG. 4D shows physicochemical properties of the Fe/KA material.

FIG. 5A is a bar graph showing the amount of captured CO₂.

FIG. 5B is a graph showing hydrogenation of captured CO₂ during the five cycles of FIG. 5A.

FIG. 6A is a graph showing a comparison of gas-phase hydrogenation at 320° C.

FIG. 6B is a graph showing a comparison of gas-phase hydrogenation at 360° C.

FIG. 6C is a graph showing a comparison of XRD of Fe/KA for direct air capture and gas-phase reactions carried out at 1:10 and 1:3 ratios of CO₂/H₂.

FIG. 7 is a scheme showing a proposed mechanism for the conversion of captured CO₂ to hydrocarbons.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Current direct air capture (DAC) approaches require a significant amount of energy for heating CO₂-sorbed materials for regeneration and for compressing CO₂ for transportation purposes. Rationally designing materials offering both capture and conversion functionalities can lead to more energy- and cost-efficient direct air capture and conversion. The instant disclosure provides a single sorbent-catalytic (non-noble metal) material that can be used for the Integrated Direct Air Capture and CATalytic (iDAC-CAT) conversion of captured CO₂ into value-added products.

Given the increasing CO₂ concentration in the atmosphere, rapid and massive deployment of negative emission technologies (NETs) will be needed to limit global temperature increase to 1.5-2° C. In general, NETs should be large enough to remove several gross tons (Gt) of CO₂ from the atmosphere and in this context, direct air capture is expected to complement other NET options. A variety of sorbents have been investigated for CO₂ capture, including physisorbents such as metal-organic frameworks, zeolites, and activated carbon, as well as chemisorbents such as amine-functionalized adsorbents that commonly contain polyamines. Particularly, chemisorbents are suited for CO₂ capture from ultra-dilute sources such as air due to strong chemical interactions between CO₂ and sorbents. As a result, chemisorbents are the subject of understanding and improving their CO₂ adsorption and desorption processes. However, the economic feasibility of large-scale deployment of current direct air capture systems is uncertain due to high energy input needed for the desorption process (cost estimates are $200-1000/ton CO₂ for direct air capture compared to $36-53/ton CO₂ for coal-derived flue gas). Currently, there are no commercially relevant technologies that can economically produce either value-added fuels and chemicals, or solid products for storage using CO₂ captured from air.

The CO₂ capture and CO₂ conversion has long been viewed as two independent processes. The direct conversion of captured CO₂ into value-added products (coupled approach) is superior to traditional decoupled CO₂ capture and CO₂ conversion because the coupled approach avoids the energy-intensive sorbent regeneration (CO₂ desorption), compression and transportation steps. New reactive pathways for the CO₂ conversion can be realized in the capture media, leading to higher conversion, selectivity, and reduced cost. For example, typical gas-phase CO₂ hydrogenation to methanol requires high temperatures due to slower kinetics. At high temperature, a competing reaction—the reverse water gas shift reaction—is also favored, which reduces the selectivity and consumes valuable H₂. On the other hand, in the amine-based capture medium, CO₂ hydrogenation to methanol followed a nontraditional route for conversion to methanol through a formamide intermediate. This nontraditional low-temperature methanol synthesis route was made possible by the presence of an amine-based capture medium. However, amine-based aqueous/non-aqueous solvents are not suitable for direct air capture application due to high volatility, viscosity, and evaporative loss of water under realistic direct air capture conditions. For direct air capture, solid sorbents have several benefits (over well-studied liquid sorbents) such as increased adsorption capacities, lower regeneration energy penalties, relative ease of handling, and improved recyclability.

Though the feasibility of integrating capture and conversion processes has been shown with liquid systems, the material design principles are not transferable to solids because unlike liquid systems, the sorbent and catalyst need to be integrated into a single multifunctional material in solids. The solid-state iDAC-CAT approach is limited by the lack of design parameters for this multifunctional material with the cooperative sorbent and catalytic features to perform both capture and conversion. In traditional direct air capture approaches, solid or liquid sorbents with low reaction enthalpy, high capture capacity, and rapid kinetics are preferred. The strong binding of CO₂ via chemisorption is considered a limitation in traditional direct air capture approaches due to regeneration requirements. But in the iDAC-CAT approach, the strong binding can be favorable because the captured CO₂ is undergoing chemical conversion. The strong CO₂ binding can enhance the CO₂ uptake kinetics, which is important for direct air capture application.

Solid materials with dual functionalities have been used for integrated CO₂ capture and conversion to methane. Most of these materials are composed of sorbents (metal oxides and carbonates) and metal catalysts (such as Ru, Ni, and Rh). In a first step, the sorbent reacts with CO₂ to form bi(carbonate) and in a second step, (bi)carbonate reacts with hydrogen at high temperature (>300° C.) to form methane. Most of these materials also require high temperature for capture, which is not an economical option.

In this disclosure, different combinations of catalytic components and sorbents are disclosed to develop a single material that can capture CO₂ from the air at ambient conditions and then convert the captured CO₂ into valuable C₂ products such as olefins. Olefins are building blocks to produce fuels, plastics, paints, lubricants, and surfactants. Fe-based catalytic components were incorporated into the sorbent materials to facilitate the formation of C—C bonds.

Generally, direct air capture of carbon dioxide using a carbonate sorbent is a process designed to remove CO₂ directly from the atmosphere. This process begins by drawing air to the sorbent. As an example, air can be drawn in with large fans. The air, containing atmospheric concentrations of CO₂, is then directed through a contactor structure housing the carbonate sorbent.

In some examples, the ambient air can be supplemented with water (liquid or vapor). Supplementing ambient air with water in direct air capture processes can significantly enhance the efficiency and effectiveness of CO₂ removal. Water plays a role in the chemical reactions involved in carbonate-based direct air capture systems. When ambient air is passed through the contactor containing the carbonate sorbent, the presence of water facilitates the formation of bicarbonate, which is the key step in capturing CO₂. The reaction between CO₂ and the carbonate requires water to form bicarbonate. By ensuring an adequate supply of water, the reaction kinetics can be optimized, potentially increasing the rate and capacity of CO₂ absorption. Additionally, maintaining proper humidity levels in the air stream can prevent the drying out of the sorbent solution, which could otherwise reduce its effectiveness. Water also plays a role in the regeneration process, where heat is applied to release the captured CO₂ and regenerate the carbonate sorbent. Adding water also allows for the direct air capture system to be used in arid environments where the ambient air lacks humidity.

As the air flows through the contactor, a chemical reaction occurs between the CO₂ and the carbonate solution. This reaction transforms the carbonate into bicarbonate, effectively capturing the CO₂ from the air. The process can be represented by the chemical equation: CO₂+H₂O+CT₂CO₃→2CTHCO₃. As used herein “CT” refers to the cation of the carbonate. Once the sorbent becomes saturated with CO₂, it undergoes a regeneration process. This typically involves heating the solution, which reverses the absorption reaction and releases concentrated CO₂. The regeneration reaction can be expressed as: 2CTHCO₃→CT₂CO₃+H₂O+CO₂.

Following regeneration, the released CO₂ can be captured, purified, and compressed for storage or utilization or converted by a catalyst to (C₁-C₁₀)hydrocarbons described herein. Meanwhile, the carbonate solution can is cooled and recycled back to the contactor for reuse in capturing more CO₂. This process can operate continuously, with air constantly being drawn in and CO₂ being captured and released.

To achieve perform direct air capture and produce the desired products, a multifunctional material for integrated direct air capture and catalytic conversion of CO₂ is used. The multifunctional material includes a solid inorganic sorbent and a metal catalyst component integrated into a single material.

The solid inorganic sorbent is formed from a carbonate. The carbonate is at least 95 wt % and more commonly 100 wt % of the solid inorganic sorbent. The carbonate materials that can be used include calcium carbonate and magnesium carbonate as well as sodium carbonate, potassium carbonate, lithium carbonate, ammonium carbonate, and various transition metal carbonates.

Calcium carbonate primarily exists in two polymorphic forms: calcite and aragonite. Calcite, the more stable form at standard temperature and pressure, crystallizes in the trigonal-rhombohedral crystal system. Its structure consists of alternating layers of calcium ions and carbonate groups. Each calcium ion is coordinated with six oxygen atoms from different carbonate groups, forming a distorted octahedral arrangement. The carbonate groups are planar and oriented perpendicular to the c-axis of the crystal. This structure gives calcite its characteristic rhombohedral cleavage and optical properties.

Aragonite, the metastable polymorph of calcium carbonate, crystallizes in the orthorhombic system. In this structure, the calcium ions are coordinated with nine oxygen atoms from six different carbonate groups, resulting in a more densely packed arrangement compared to calcite. The carbonate groups in aragonite are slightly distorted from their planar configuration, contributing to the crystal's unique properties.

Magnesium carbonate, also known as magnesite, typically crystallizes in the trigonal-rhombohedral system, similar to calcite. However, the smaller size of the magnesium ion compared to calcium results in some structural differences. In magnesite, each magnesium ion is coordinated with six oxygen atoms from six different carbonate groups, forming a more regular octahedral arrangement than in calcite. The carbonate groups maintain their planar configuration and are oriented perpendicular to the c-axis of the crystal.

Both calcium and magnesium carbonates can form hydrated structures. For example, calcium carbonate can form ikaite (CaCO₃·6H₂O) under specific conditions, while magnesium carbonate can form various hydrates such as nesquehonite (MgCO₃·3H₂O) and lansfordite (MgCO₃·5H₂O). These hydrated forms have more complex crystal structures due to the incorporation of water molecules into the crystal lattice.

Potassium carbonate (K₂CO₃) is a white, hygroscopic salt that forms strongly alkaline solutions in water. Its crystal structure belongs to the monoclinic crystal system, specifically the P21/c space group. In this structure, the potassium ions are coordinated with oxygen atoms from the carbonate groups, forming a three-dimensional network. The carbonate ions in the crystal are planar and arranged in a way that maximizes the separation between the negatively charged oxygen atoms.

In the context of direct air capture, potassium carbonate serves as an effective sorbent for CO₂ removal from ambient air. When used in direct air capture systems, an aqueous solution of potassium carbonate reacts with atmospheric CO₂ to form potassium bicarbonate (KHCO₃). This reaction can be represented as: K₂CO₃+CO₂+H₂O→2KHCO₃. The process is reversible, allowing for regeneration of the sorbent and release of concentrated CO₂ through heating. Potassium carbonate's high solubility in water, strong alkalinity, and favorable reaction kinetics with CO₂ make it a promising candidate for large-scale direct air capture applications.

The carbonate or a mixture of the aforementioned carbonates, can be supported on a porous material of mixture of porous materials. Porous materials comprising Al₂O₃, Carbon, SiO₂, ZrO₂, aluminosilicate zeolites, or mixtures thereof are a diverse group of substances with high surface area and internal void spaces. These materials are characterized by their network of interconnected pores, which can vary in size from nanometers to micrometers. Alumina (Al₂O₃) porous materials are widely used as catalysts and adsorbents, known for their high thermal stability and surface acidity. Porous carbon materials, such as activated carbon (AC), have favorable adsorption properties due to their high surface area and tunable pore structure. Silica (SiO₂) porous materials, including mesoporous silica, are valued for their uniform pore sizes and versatility in functionalization. Zirconia (ZrO₂) porous materials offer high chemical and thermal stability, making them suitable for harsh environments. Aluminosilicate zeolites are crystalline materials with well-defined pore structures, widely used in catalysis and molecular sieving. Mixtures of these materials can combine their individual properties to create porous composites with enhanced performance for specific applications, such as gas separation, water purification, or heterogeneous catalysis.

The pore size and structure of these materials significantly influence their performance for CO₂ capture. Smaller pore sizes, typically in the microporous range (less than 2 nm), are generally more effective for CO₂ adsorption due to stronger interactions between the gas molecules and the pore walls. However, excessively small pores can limit diffusion and accessibility, reducing overall capture efficiency.

Mesoporous materials (pore sizes 2-50 nm) often provide a balance between adsorption capacity and diffusion kinetics. They allow for faster mass transfer of CO₂ molecules while still maintaining a high surface area for adsorption. The interconnectivity of pores is also crucial, as it affects the accessibility of internal surfaces and the overall capture kinetics.

The pore structure's uniformity can impact selectivity for CO₂ over other gases. Well-defined pore structures, such as those in zeolites, can act as molecular sieves, preferentially adsorbing CO₂ based on size exclusion principles.

The metal catalyst is used to synthesize the (C₁-C₁₀)hydrocarbyl. The metal catalyst can include iron, cobalt, copper, manganese or any combination of oxides thereof. These transition metals are useful as catalysts because of their ability to form multiple oxidation states, coordinate and activate CO₂, H₂ and bicarbonates through their vacant d-orbitals, and stabilize CO₂-derived intermediates. The catalytic process, generally, includes several steps. First, the metal catalyst activates CO₂ by weakening its C═O bonds, making the carbon more reactive. Next, the activated CO₂ undergoes reduction, often through electron transfer from the metal catalyst or a reducing agent. Finally, the reduced carbon species couple with another activated carbon to form a C—C bond. The specific mechanisms vary depending on the metal and reaction conditions. For instance, iron catalysts often operate through a Fischer-Tropsch-like process, while cobalt catalysts can facilitate reductive dimerization of CO₂ to form oxalate intermediates. Copper catalysts are known for reducing CO₂ to hydrocarbons and alcohols, with C—C bond formation occurring through coupling of intermediate species. Manganese complexes have demonstrated the ability to catalyze the reductive coupling of CO₂ to form oxalate and other C2 products. The efficiency and selectivity of these processes can be influenced by factors such as the metal's oxidation state, ligand environment, reaction conditions, and the presence of co-catalysts or promoters. The metal catalyst particles can range from about 2 wt % to about 50 wt % of the material, 20 wt % to about 30 wt %, less than, equal to, or greater than about 2 wt %, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt %.

Captured CO₂ are converted to (C₁-C₁₀)hydrocarbyl products upon hydrogenation. Suitable examples of (C₁-C₁₀) hydrocarbyl products include olefins, paraffins, or both. C₁-C₁₀ olefins and paraffins are important classes of hydrocarbons widely used, for example, in the petrochemical industry. Olefins, also known as alkenes, are unsaturated hydrocarbons containing at least one carbon-carbon double bond. Examples include ethylene (C₂) to decene (C₁₀), with examples including propylene, butene, and pentene. These compounds are highly reactive due to their double bond, making them valuable feedstocks for producing plastics, synthetic rubbers, and other chemical products.

Paraffins, also called alkanes, are saturated hydrocarbons with single bonds between carbon atoms. Examples include methane (C₁) to decane (C₁₀), including compounds like ethane, propane, and butane. Paraffins are less reactive than olefins and are commonly used as fuels, solvents, and in the production of various petrochemicals.

The material described herein is capable of a high degree of selectivity in the products formed. For example, the material is capable of converting captured CO₂ to (C₁-C₁₀)hydrocarbyls with a selectivity of at least 10% for C₂-C₄ olefins or paraffins, at least 15%, at least 20%, at least, 25%, at least 30%, at least 35%, at least 40%, at least 45%, at least 50%, at least 55%, at least 60%, at least 65%, at least 70%, at least 75%, at least 80%, at least 85%, at least 90%, or at least 95% for C₂-C₄ olefins or paraffins.

The disclosed material is capable of converting captured CO₂ to (C₁-C₁₀)hydrocarbyls at temperatures in a range of from about 250° C. to about 400° C., 330° C. to about 360° C., less than, equal to, or greater than about 250° C., 260, 265, 270, 275, 280, 285, 290, 295, 300, 305, 310, 315, 320, 325, 330, 335, 340, 345, 350, 355, 360, 365, 370, 375, 380, 385, 390, 395, or about 400° C. The concentration of CO₂ in the air contacted with the material can range from about 350 ppm CO₂ to about 1000 ppm CO₂, about 500 ppm CO₂ to about 800 ppm CO₂, less than, equal to, or greater than about, 350 ppm CO₂, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 ppm CO₂. Additionally, the material is capable of capturing CO₂ from concentrated sources containing about 1 vol % CO₂ to about 30 vol % CO₂, about 5 vol % CO₂ to about 20 vol % CO₂, less than, equal to, or greater than about 1 vol % CO₂, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 vol % CO₂. The material can have a CO₂ capture capacity in a range of from about 1.5 wt % to about 25 wt %, about 5 wt % to about 20 wt %, less than, equal to, or greater than about 1.5 wt %, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or about 25 wt % at temperatures in a range of from about ambient temperatures to about 150° C. in the presence of water vapor. Ambient air temperature refers to the temperature of the surrounding air in a given environment, typically measured outdoors and away from direct heat sources. It represents the general temperature conditions of the atmosphere at a specific location and time.

As demonstrated further in the Examples section, a particularly suitable construction is a potassium carbonate sorbent disposed on an aluminum oxide scaffold having iron particles impregnated thereon. The material can be represented by the formula: Fe/K₂CO₃/Al₂O₃.

### Examples

Various aspects of the present disclosure can be better understood by reference to the following Examples which are offered by way of illustration. The present disclosure is not limited to the Examples given herein.

CO₂ Capture Studies Using K₂CO₃/Al₂O₃

Inorganic chemisorbents are chosen for this because they are more durable and low-cost materials compared to amine-based sorbents for direct air capture. The commonly used inorganic chemisorbents for direct air capture are CaO, MgO, and alkali metal carbonates. Among these sorbents, alkali metal carbonates can perform capture at ambient temperature. To increase the carbonation rate of alkali carbonates, they are usually dispersed on high-surface-area materials such as alumina, Al₂O₃. In this Example, 25 wt % of K₂CO₃/Al₂O₃ was synthesized, characterized, and evaluated at 25° C. at different capture conditions to identify suitable conditions for direct air capture. As-synthesized K₂CO₃/Al₂O₃ was characterized by BET analysis. Type IV isotherms with a characteristic hysteresis loop for both Al₂O₃ and K₂CO₃/Al₂O₃ were realized in the BET analysis, indicating that alumina is mesoporous in nature. Impregnation of K₂CO₃ over alumina resulted in a decrease in both surface area and pore volume of the original support, but the average pore sizes were almost comparable. This implies that smaller sizes of K₂CO₃ filled the pores of the mesoporous alumina, confirming the dispersion of K₂CO₃ over the alumina surface.

The effect of pretreatment conditions and water vapor content on the capture performance of the sorbent was studied. The K₂CO₃/Al₂O₃ sorbent was first pretreated at 200° C. for 1 h under N₂ flow (100 mL/min.). The material was then cooled to room temperature and pre-saturated with both 0.5 and 1.0 mol % H₂O vapor, followed by introduction of 400 ppm of CO₂. The amount of CO₂/g of sorbent adsorbed during both the experiments was calculated from the molar flow concentration profile of CO₂ versus time. For 0.5 mol % of H₂O, 850 mmol/g of CO₂ was adsorbed, whereas in the case of 1 mol % of H₂O, 770 mmol/g of CO₂ was adsorbed, showing that the 0.5 mol % of H₂O had a slightly higher adsorption capacity.

Next, CO₂ was co-fed with 0.5 mol % H₂O vapor over the pretreated K₂CO₃/Al₂O₃ and compared with the pre-saturated sample. The water vapor co-fed sample shows the highest sorption capacity of 6.5 wt % compared to the water vapor pretreated samples, which is also visible from the area under the curve for the water vapor co-fed and pretreated. The amount of CO₂ adsorbed by the 25 wt % K₂CO₃/Al₂O₃ sorbent is ˜6.5 wt % higher than the amount reported in the literature, which are 3.6 wt % for K₂CO₃/Al₂O₃ and 4.1 wt % for K₂CO₃/Al₂O₃-700 (Al₂O₃ heated at 700° C. before K₂CO₃ impregnation).

X-ray diffraction (XRD) analysis shows the change in phase composition of the K₂CO₃/Al₂O₃ before and after air capture at r.t in the presence of water vapor. For fresh K₂CO₃/Al₂O₃, the main diffraction peaks were attributed to dawsonite, KAlCO₃(OH)₂, K₂CO₃, and g-Al₂O₃. The formation of the dawsonite on the fresh samples takes place due to the exposure of as-synthesized K₂CO₃/Al₂O₃ to air. This agrees with the TPD of the fresh material, where the peak at 350° C. is due to the decomposition of the dawsonite.

Thermal decomposition of the air-captured K₂CO₃ using TPD shows two characteristic peaks within 100-200° C., which is likely due to the decomposition of the species containing bicarbonate, K₂CO₃·2KHCO₃·1.5H₂O, and KHCO₃. This agrees with the XRD diffraction patterns of the air-captured sorbent. The higher-temperature peak is mainly due to the decomposition of the KAlCO₃(OH)₂, which was reported to take place between 26° and 320° C.

K₂CO₃/AC was also synthesized and tested for CO₂ capture because activated carbon was also known to be a suitable support material. Compared to K₂CO₃/Al₂O₃, the capture capacity of K₂CO₃/AC was 1.3 times lower. Due to the superior capture performance of K₂CO₃/Al₂O₃ under the optimized reaction conditions, it was chosen as the sorbent material for the integrated capture and conversion studies.

Conversion of Captured CO₂ to C₁ and C₂Products

The direct conversion of captured CO₂ from air or concentrated point sources to C₁ products such as methane, methanol, and CO has been demonstrated. However, due to the high energy barrier of C—C coupling reactions, conversion of captured CO₂ to C₂₊ products is still a challenge. Combining the endothermic reverse water gas shift (CO₂+H₂→CO+H₂O) reaction with the exothermic Fischer-Tropsch (CO+H₂→CₓH_{y}) reaction has been identified as one of the strategies for converting concentrated streams of CO₂ and H₂ in the gas phase to C₂₊ products. Particularly, potassium (alkali metal) modified Fe-based catalysts are known to promote carbon-chain growth in the gas-phase CO₂ hydrogenation reaction. It was hypothesized that by combining the Fe-based catalysts and potassium-based sorbents, the captured CO₂ can be directly converted to C₂₊ products, bypassing the energy-intensive CO₂ regeneration and compression steps. To test this, different combinations of iron and K₂CO₃/Al₂O₃ based sorbent-catalytic materials were synthesized and evaluated the capture and conversion performance of these synthesized materials.

A) Fe₂O₃—K₂CO₃/Al₂O₃

A physical mixture of Fe₂O₃ and K₂CO₃ has been demonstrated to be effective at converting CO₂ into C₂-C₄ olefins with a selectivity of about 31% via a tandem mechanism. The addition of K₂CO₃ is used for promoting the formation of CO (via potassium bicarbonate and potassium formate intermediates), which gets converted to olefins and paraffins in the presence of iron oxide and iron carbide phases at 350° C. Based on this, a physical mixture of Fe₂O₃—K₂CO₃/Al₂O₃ (Fe₂O₃-KA). The Fe₂O₃-KA was prepared and pretreated at 400° C. under H₂ flow (100 mL/min) for 1 h to convert Fe₂O₃ to Fe nanoparticles. CO₂ capture was performed using 400 ppm of CO₂ (1200 mL/min) with 0.5 mol % of H₂O at 25° C. The capture performance was compared with K₂CO₃/Al₂O₃, which was activated under similar conditions. Under this condition, ˜100% of the K₂CO₃ was utilized during CO₂ capture in the case of K₂CO₃/Al₂O₃, whereas in the case of Fe₂O₃—K₂CO₃/Al₂O₃, only 81% of the K₂CO₃ was utilized in CO₂ capture. High-temperature pretreatment enhanced the capture capacity through the dawsonite decomposition reaction. Then, hydrogenation of the captured CO₂ was performed under hydrogen pressure of 145 psi at 320° C. (hold for 2.5 h) and 360° C. (hold for 2 h) at a ramp rate of 5° C./min under H₂ flow (60 mL/min). This resulted in desorption of CO₂ with no detectable amount of hydrogenated CO₂-derived products. Most of the CO₂ was released at ˜320° C., suggesting that dawsonite is the major species formed during CO₂ capture.

B) K₂CO₃—Fe/C and K₂CO₃—Fe/C/Al₂O₃

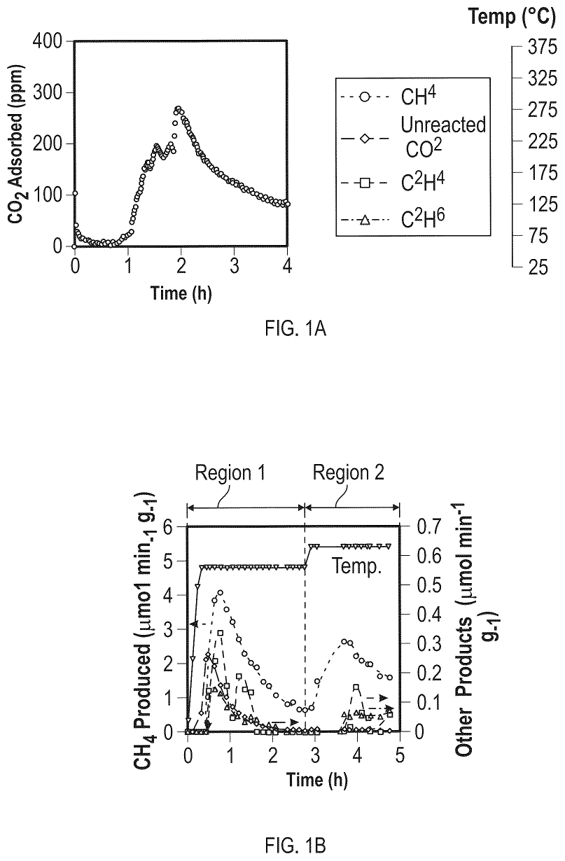

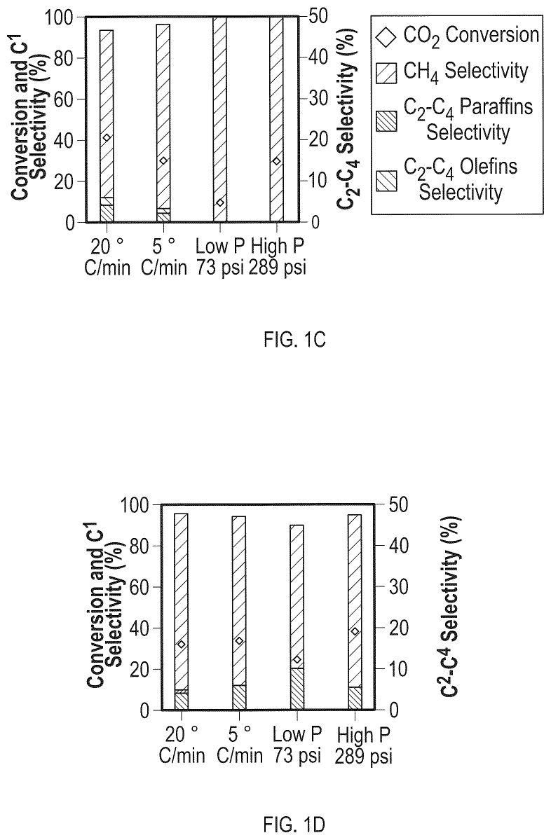

The use of potassium-promoter-modified Fe/C catalysts can increase olefin selectivity in CO₂ hydrogenation. Fe/C was synthesized by the hydrothermal method. K₂CO₃/Fe/C was formed by impregnating K₂CO₃ (25 wt %) on the Fe/C catalyst. The synthesized material was pretreated at 400° C. under H₂ flow for 10 h to ensure carbide formation before CO₂ capture and conversion studies. CO₂ capture was performed by following a standard capture procedure. The capture profile is shown in FIG. 1A. In the first 50 min, there was an induction period after which the CO₂ capture breakpoint started. The initial delay could either be due to physical adsorption of the CO₂ occupying the macropores of the materials or because the material surface was not immediately saturated with water vapor, which is necessary to start the carbonation reaction. The total CO₂ captured in 4 hours by this material typically ranges between 600 and 700 mmol/g, which is ˜2 times lower than that of K₂CO₃/Al₂O₃(Table 1). The reduction of the captured CO₂ was carried out under H₂ flow of 60 mL/min under four different conditions: (1) heating rate of 5° C./min to 320° C. (hold for 2.5 h) and 360° C. (hold for 2 h) at 145 psi, (2) heating rate of 20° C./min to 320° C. (hold for 2.5 h) and 360° C. (hold for 2 h) at 145 psi, (3) heating rate of 5° C./min to 320° C. (hold for 2.5 h) and 360° C. (hold for 2 h) at 73 psi, and (4) heating rate of 5° C./min to 320° C. (hold for 2.5 h) and 360° C. (hold for 2 h) at 289 psi. FIG. 1B shows the hydrogenation results of the captured CO₂. With the increase in temperature, some unreacted CO₂ started to desorb. Along with CO₂, CH₄ also formed and was the highest when the temperature reached 320° C. C₂H₄ and C₂H₆ were also produced at Region 1 (at 320° C., 2.5 h). Further increasing the temperature to 360° C. resulted in additional CH₄ production along with small amounts of ethylene and ethane. There were no detectable amounts of higher olefins or paraffins formed. Increasing the heating rate from 5 to 20° C./min increased the overall conversion of CO₂ to methane along with a slight increase in olefin selectivity (2%) at 320° C. The profile for the time-dependent conversion and yield of products is shown in FIG. 1B. With additional increase in the temperature to 360° C., ˜74% of the total captured CO₂ was converted to C₁ and C₂ products with ˜94.4% selectivity to methane, 4.2% selectivity to ethane, and 1.4% selectivity to ethene. This is the first demonstration of the conversion of captured CO₂ (air derived) to C₁ and C₂ based products in the presence of an Fe-based catalyst. Besides the formation of olefins, which are the target products, the production of renewable methane from the captured 400 ppm CO₂ is also a great advantage. This can contribute to an alternative for natural gas, and its direct use in existing infrastructure could result in a lower carbon footprint. Varying the hydrogen pressure for the conversion further improved the selectivity to methane with a decrease in the conversion of the captured CO₂ (FIGS. 1C and 1D).

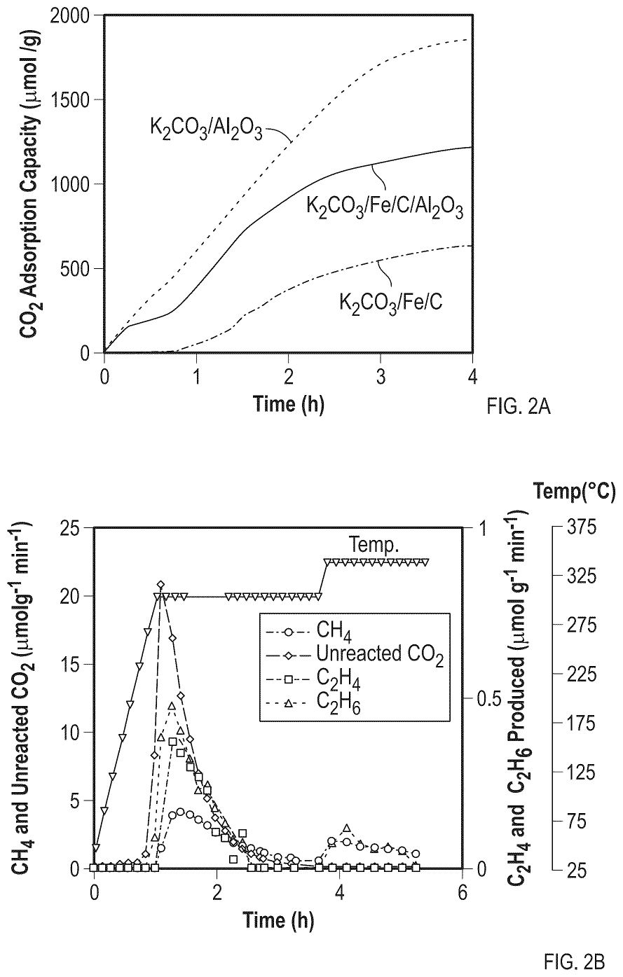

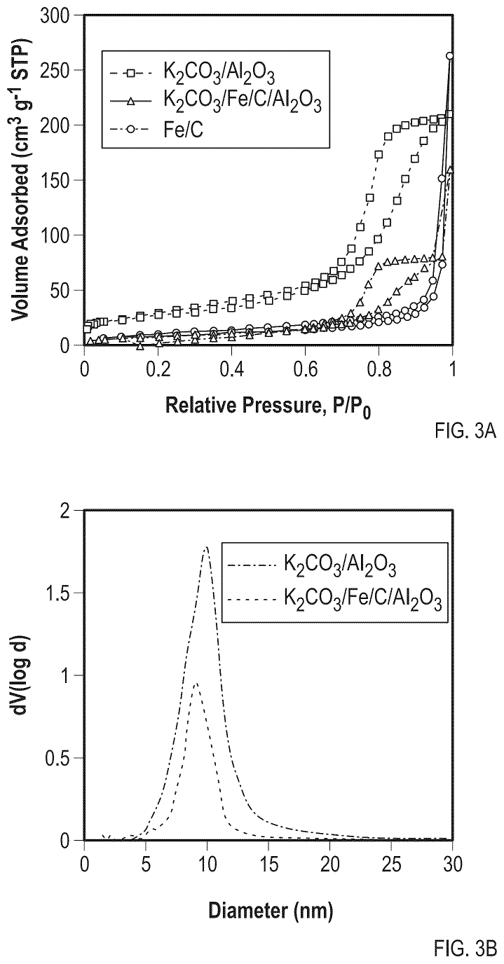

A decrease in CO₂ capture with K₂CO₃/Fe/C compared to K₂CO₃/Al₂O₃ is likely due to the smaller surface area of Fe/C (33.16 m²/g), which results in larger K₂CO₃ particles (Table 1). Lower CO₂ loading could inhibit C—C bond formation because there are fewer carbons. To increase the surface area and eventually improve the capture performance, K₂CO₃/Fe/C/Al₂O₃ was synthesized via the wet impregnation method and the adsorption capacity was compared with that of K₂CO₃/Al₂O₃ and K₂CO₃/Fe/C under similar capture conditions. The capture performance was significantly improved after the addition of Al₂O₃. The K₂CO₃/Fe/C/Al₂O₃ captured ˜1220 mmol/g of CO₂ (vs. 600-700 mmol/g of CO₂ for K₂CO₃/Fe/C) (FIG. 2A). This difference can be explained from the BET results of the support over which K₂CO₃ was impregnated. The BET isotherms of three materials are shown in FIG. 3A. As discussed earlier, dispersion of K₂CO₃ on Al₂O₃ retained the mesoporosity of the support and showed a type IV isotherm despite a decrease in the surface area as shown in Table 1.

The isotherm of Fe/C is a type II isotherm with no pronounced hysteresis loop, showing that the material is either non-porous or macroporous. The surface area is very low compared to the Al₂O₃ support and has no pores, as shown in Table 1. Therefore, the impregnation of K₂CO₃ could have formed larger particles on Fe/C, leading to lower CO₂ capture. Due to the presence of the Al₂O₃ pores, K₂CO₃ was well dispersed over a mixture of high-surface-area, mesoporous Al₂O₃ and non-porous Fe/C. This led to higher CO₂ capture for K₂CO₃/Fe/C/Al₂O₃ compared to only K₂CO₃/Fe/C, as shown in Table 1.

TABLE 1
Characterizations of the materials from the N₂ adsorption isotherm.

| | | | | | | Catalytic Activity | | | | |
|---|---|---|---|---|---|---|---|---|---|---|
| | Physical Properties | | | | | | | C₂-C₄ | C₂-C₄ | |
| | | | Average | | | CO₂ | CH₄ | Paraffins | Olefins | C₅₊ |
| | SA | PV | Diameter | CO₂ Capture | | Conv. | Sel | Sel | Sel | Sel |
| Materials | (m²/g) | (cm³/g) | (nm) | (μmol/g) | (wt %) | (%) | (%) | (%) | (%) | (%) |
| Fe/C | 33.16 | 0.4008 | N/A | N/A | N/A | N/A | N/A | N/A | N/A | N/A |
| Al₂O₃ | 182.4 | 0.6001 | 11.4 | N/A | N/A | N/A | N/A | N/A | N/A | N/A |
| K₂CO₃/Al₂O₃ | 99.19 | 0.3262 | 10.09 | 1862 | 8.2 | N/A | N/A | N/A | N/A | N/A |
| K₂CO₃/Fe/C ^{a} | — | — | — | 600-700 | 2.6-3.1 | 30.0 | 96.8 | 2.2 | 1.0 | 0.0 |
| K₂CO₃/Fe/C ^{b} | — | — | — | | | 41.4 | 93.9 | 4.1 | 2.0 | 0.0 |
| K₂CO₃/Fe/C/ | 29.23 | 0.2476 | 8.95 | 1223 | 5.4 | 30.5 | 83.2 | 8.6 | 7.3 | 0.9 |
| Al₂O₃ ^{a} | | | | | | | | | | |

^{a} Heating rate at 5° C./min during hydrogenation of captured CO₂,
^{b} heating rate of 20° C./min
Pretreatment: H₂ 60 mL/min, 400° C., 5 h
CO₂ capture: CO₂ 400 ppm, 1200 mL/min, H₂O vapor 0.5 mol %, 25° C., 4 h
Conversion and selectivity at 320° C. under H₂ flow (60 mL/min) and 145 psi

With the improvement in the capture performance, the CO₂ captured in K₂CO₃/Fe/C/Al₂O₃ was hydrogenated in situ (FIG. 2B shows the hydrogenation profile of captured CO₂). A comparison of the hydrogenation activities of the K₂CO₃/Fe/C and K₂CO₃/Fe/C/Al₂O₃ is shown in Table 1. Interestingly, the C₂-C₄ olefin selectivity significantly improved to 7.3% in the case of K₂CO₃/Fe/C/Al₂O₃. The improved C—C coupled products formation could be because of the relatively high CO₂ loading. In addition, a small amount of C₅₊ products (˜1%) was also detected. Increasing the hydrogenation temperature to 360° C. only improved the conversion and selectivity further to methane. Increased methane formation at higher temperature could be due to less carbon (i.e., captured CO₂) content on the material, which could prevent C—C formation.

C) Fe/K₂CO₃/Al₂O₃ and Fe—CO/K₂CO₃/Al₂O₃

Because the physical mixture of Fe₂O₃—K₂CO₃/Al₂O₃ formed no CO₂ hydrogenation products, we prepared Fe/K₂CO₃/Al₂O₃ (Fe/KA) and Fe—CO—K₂CO₃/Al₂O₃ (Fe—CO/KA) (by incipient wetness impregnation of Fe and/or Co salts on K₂CO₃/Al₂O₃) to improve the cooperativity between Fe and K to produce C—C products. After pretreating these materials at 400° C. for 5 h under H₂ flow, the CO₂ capture was performed under standard conditions (400 ppm of CO₂, 0.5 mol % of H₂O, 25° C., 4 h). The Fe—CO/KA captured 1970 mmol/g of CO₂, which is almost similar to K₂CO₃/Al₂O₃ (pretreated at 400° C.), showing that the addition of the catalytic component had no impact on the capture performance. Hydrogenation of the captured CO₂ using Fe—CO/KA was carried out at two different temperature ramp rates, 5 and 20° C./min. Increasing the heating rate decreased the CO₂ conversion to value-added products with no significant impact on product distribution, as shown in Table 2.

TABLE 2
Comparison of CO₂ capture and conversion for Fe—Co/
K₂CO₃/Al₂O₃ and Fe/K₂CO₃/Al₂O₃ at 320° C.

| | Heating | | | | | |
|---|---|---|---|---|---|---|
| | Rate | CO₂ | CO₂ | | C₂-C₄ | C₂-C₄ |
| | (° C./ | Captured | Conv. | CH₄ | Paraffins | Olefins |
| | min) | (mmol/g) | (%) | Sel(%) | Sel (%) | Sel (%) |
| Fe—Co/ | 5 | 1970 | 21.3 | 88.3 | 5.1 | 6.7 |
| KA | | | | | | |
| | 20 | 1970 | 12.0 | 86.8 | 8.3 | 4.9 |
| Fe/KA | 5 | 1645 | 22.4 | 79.7 | 8.9 | 11.4 |

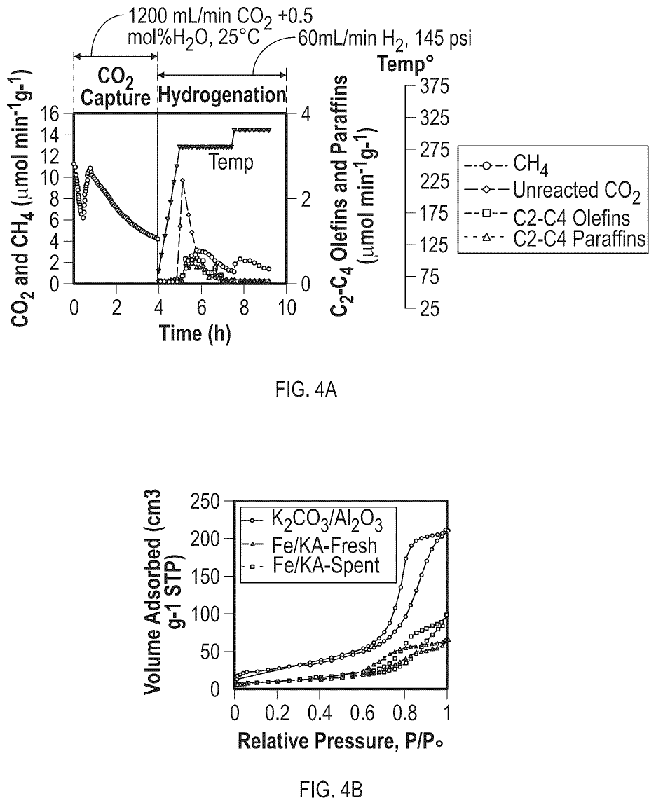

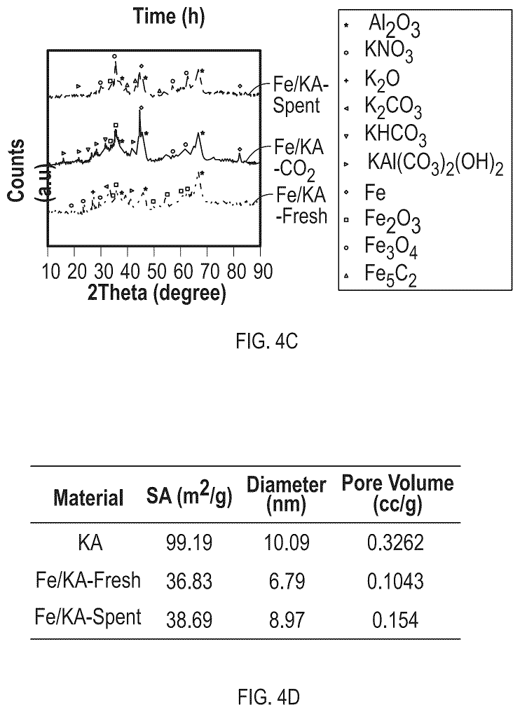

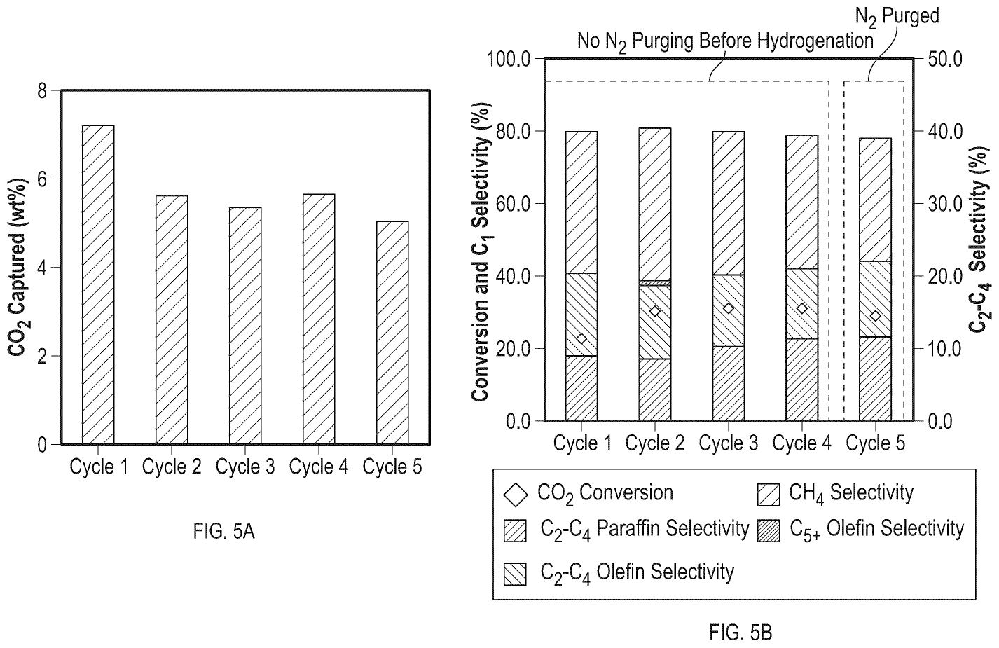

The Fe/KA captured ˜1700 mmol/g of CO₂ at our standard capture conditions. The hydrogenation results are shown in FIG. 4A and Table 2. At 320° C., C₂-C₄ olefins and paraffins started forming accompanied with the formation of CH₄. The highest olefin selectivity of ˜11.4% was obtained with a CO₂ conversion of 22.4%. The BET isotherm shows that the mesoporosity of the K₂CO₃/Al₂O₃ is still maintained after impregnation of Fe particles (FIG. 4B). Upon impregnating Fe, the surface area decreased from 99.19 (for K₂CO₃/Al₂O₃) to 36.83 m²/g and the diameter of the mesopores decreased to 6.79 nm, confirming the formation of Fe particles inside the mesopores (FIG. 4D). The average pore size of the spent Fe/KA material (after hydrogenation) increased compared to the fresh material along with slight increase in the pore volume and surface area. This shows that after the hydrogenation, more dispersed particles were formed. This could be due to the formation of Fe₅C₂ and Fe₃O₄ particles during the high-temperature hydrogenation.

FIG. 4C shows wide-angle XRD of the freshly captured CO₂ and spent Fe/KA. In the fresh sample, diffraction peaks corresponding to KNO₃, dawsonite, and Fe₂O₃ particles were evident. The fresh sample was pretreated at 400° C. under pure H₂ flow for 5 h before air capture in the presence of water vapor. XRD of the CO₂-captured Fe/K₂CO₃/Al₂O₃ material shows peaks for KHCO₃ and dawsonite along with some Fe₂O₃ and Fe particles. The Fe particles could form from Fe₂O₃ due to hydrogenation with H₂ at high temperature. The spent (after hydrogenation) Fe/KA shows peaks for Fe₅C₂ along with Fe₃O₄, which were formed during hydrogenation of the captured CO₂. The formation of these dispersed particles resulted in an increase of pore volume and of the average pore size of the material. The formation of the Fe₅C₂ phase shows the carburization of Fe₃O₄ particles.

The spent Fe/K₂CO₃/Al₂O₃ after the first cycle of capture and hydrogenation was reused to study the robustness of these materials. The capture capacity was reduced in the second cycle to 1276 mmol/g (5.6 wt CO₂%) compared to 1645 μmol/g (7.4 wt CO₂%) in the first cycle. However, the capture performance was steady in the subsequent third (5.4 wt %), fourth (5.6 wt %), and fifth (˜5.03 wt %) cycles. The drop in the capture capacity could be because of the presence of K₂O in the fresh Fe/KA, which consumed CO₂ from air to form K₂CO₃. A similar drop in the capture capacity was observed between the first (6.5 wt % CO₂) and second cycles (5.3 wt % CO₂) for K₂CO₃/Al₂O₃(Table S3). However, in this case (K₂CO₃/Al₂O₃), the drop in performance could be because the low-temperature pretreatment conditions (at 200° C. for 1 h) prevented the conversion of dawsonite back to K₂CO₃. Before hydrogenation during the fifth cycle, the CO₂-captured material was purged with N₂ flow for 1 h to quantify physiosorbed CO₂ content. Only trace amounts of CO₂ were released during the N₂ purge, and subsequent hydrogenation showed consistent conversion and selectivity to products, demonstrating that the material is stable for at least five cycles.

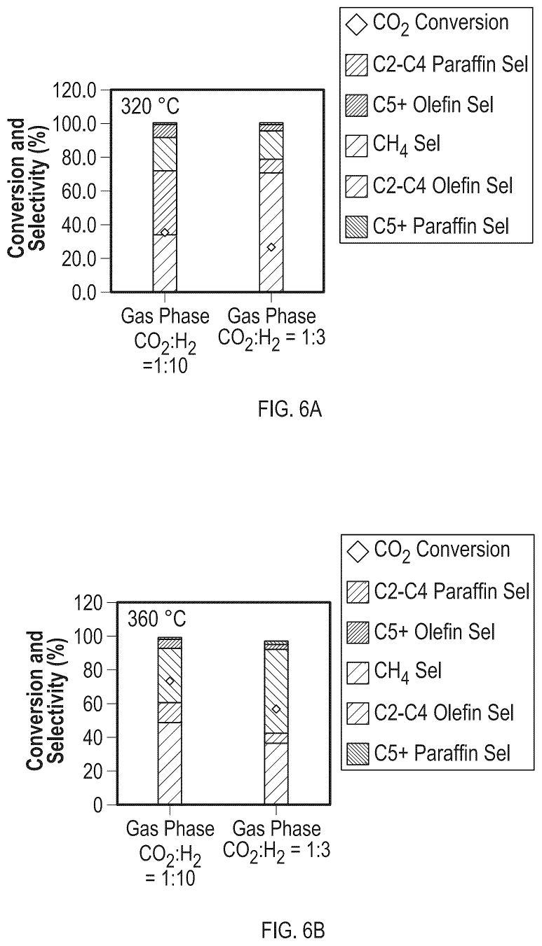

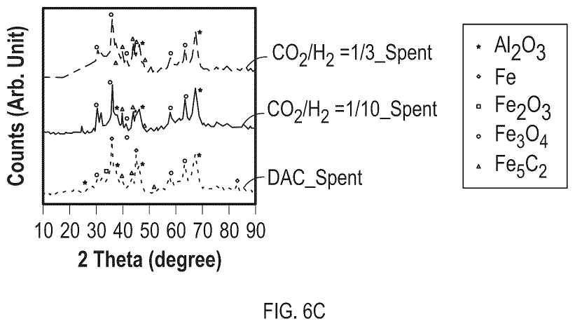

To understand the effect of the CO₂-to-H₂ ratio and reaction temperature on the product distribution and conversion, the gas-phase hydrogenation studies were performed with Fe/K₂CO₃/Al₂O₃ using 1:3 and 1:10 ratios of CO₂:H₂. The conversion results for the Fe/K₂CO₃/Al₂O₃ at 320° C. and 360° C. are shown in FIG. 6 and Tables 3 and 4. At 320° C., in the case of the 1:10 ratio of CO₂/H₂, the selectivity to C₂-C₄ paraffins was very high compared to direct air capture and 1:3 ratio of CO₂/H₂ studies. The selectivity to C₂-C₄ olefins was not significantly altered by the CO₂/H₂ ratio. In addition to C₁₋₄ products, C₅₊ products were detected by gas chromatography. The reaction temperature played a significant role in hydrogenation product selectivity and CO₂ conversion. The CO₂ conversion was 75% and 51% for 1:10 and 1:3 ratios of CO₂/H₂, respectively, at 360° C. (Table 4). High olefin selectivities (>50%) and O/P (olefin/paraffin) ratios (˜10) were achieved for a 1:3 ratio of CO₂/H₂ at 360° C.

TABLE 3
Comparison of hydrogenation of captured
CO₂ with gas-phase CO₂ at 320° C. over Fe/KA.

| | CO₂ | CH₄ | C2-C4 | C2-C4 | C5+ | C5+ | |
|---|---|---|---|---|---|---|---|
| | Conv. | Sel | Paraffins | Olefins | Olefins | Paraffins | O/P |
| | (%) | (%) | Sel (%) | Sel (%) | (Sel %) | (Sel %) | Ratio |
| DAC | 22.4 | 79.7 | 8.9 | 11.4 | 0.0 | 0.0 | 1.3 |
| 1:10 | 34.5 | 34.0 | 38.0 | 19.9 | 8.17 | 0.0 | 0.5 |
| 1:3 | 27.1 | 71.3 | 7.88 | 16.9 | 3.84 | 0.15 | 2.1 |

The XRD spectra of the spent direct air capture and gas-phase CO₂ hydrogenation materials are shown in FIG. 6C. Fe₃O₄ was observed in all spent materials. The Fe₅C₂ diffraction patterns are more pronounced for the 1:3 CO₂/H₂ reaction compared to the 1:10 CO₂/H₂ reaction. This agrees with the decreased CH₄ selectivity and increased O/P ratio of the 1:3 CO₂/H₂ reaction because both Fe₃O₄ (for the RWGS) and Fe₅C₂ are important for C—C formation. Peaks for Fe were also observed in the spent direct air capture material, showing that not all the Fe₂O₃ was converted to Fe for carburization. The formation of the carbide-phase reaction route is as follows: Fe₂O₃→Fe₃O₄→FeO→Fe, and then finally the Fe is carburized to Fe₅C₂.

TABLE 4
Comparison of hydrogenation of gas-
phase CO₂ at 360° C. over Fe/KA.

| | CO₂ | CH₄ | C2-C4 | C2-C4 | C5+ | C5+ | |
|---|---|---|---|---|---|---|---|
| | Conv. | Sel | Paraffins | Olefins | Olefins | Paraffins | O/P |
| | (%) | (%) | Sel (%) | Sel (%) | (Sel %) | (Sel %) | Ratio |
| 1:10 | 74.7 | 49.2 | 11.8 | 32.8 | 5.74 | 0.0 | 2.78 |
| 1:3 | 50.9 | 37.1 | 5.16 | 50.9 | 2.96 | 1.33 | 9.86 |

The Fourier transform infrared spectroscopy (FTIR) spectrum of the spent direct air capture and gas-phase CO₂ hydrogenation materials were compared. The C—H vibrations were seen between 2960-2627 cm⁻¹ corresponding to formate and other bound —CH species. The carbonyl vibration of formate was observed at the ˜1631 cm⁻¹ region. The Fe—CO interactions were visible in the 1800-2100 cm⁻¹ region, which corresponds to bound CO with different forms of Fe. In addition to formate and CO, there are additional bands visible for carbonates and bicarbonates in the infrared spectrum.

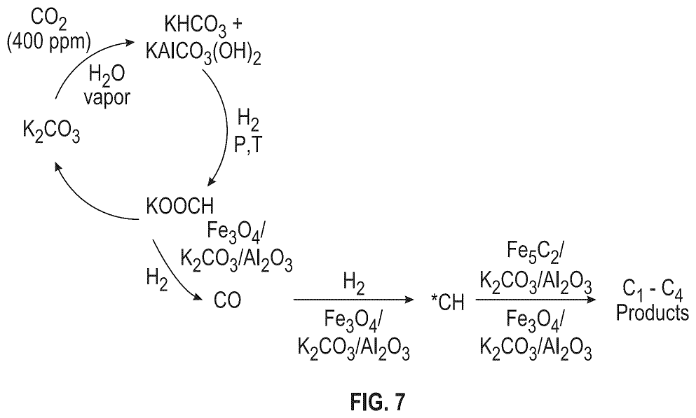

Based on the selectivity of the products and the XRD and FTIR of the spent samples, the conversion of captured CO₂ to olefins occurs via the direct CO₂ conversion pathway, where the CO₂ is converted to CO via RWGS in the presence of Fe₃O₄. The CO is then converted to olefins following the FTS mechanism in the presence of Fe₅C₂. A proposed pathway has been shown in the scheme of FIG. 7.

When CO₂ is captured (400 ppm) in the presence of water vapor at room temperature, the K₂CO₃ of Fe/K₂CO₃/Al₂O₃ transforms to KHCO₃ and KAlCO₃(OH)₂, which forms HCOOK and CO upon hydrogenation catalyzed by Fe₃O₄/K₂CO₃/Al₂O₃. The Fe₃O₄/K₂CO₃/Al₂O₃ is formed from Fe₂O₃/K₂CO₃/Al₂O₃ in the presence of H₂. The Fe₃O₄/K₂CO₃/Al₂O₃ helps further convert CO to *CH species, which undergo C—C coupling in the presence of Fe₅C₂ formed in situ during the reaction. From our experimental work, we show that the proximity between the Fe and K on the Al₂O₃ is important for CO₂ activation and conversion to C—C products.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present disclosure. Thus, it should be understood that although the present disclosure has been specifically disclosed by specific aspects and optional features, modification and variation of the concepts herein disclosed may be resorted to by those of ordinary skill in the art, and that such modifications and variations are considered to be within the scope of aspects of the present disclosure.

### Exemplary Aspects.

The following exemplary aspects are provided, the numbering of which is not to be construed as designating levels of importance:

Aspect 1 provides a multifunctional material for integrated capture and catalytic conversion of CO₂, comprising:
a solid inorganic sorbent; and
a metal catalyst component,
wherein the solid inorganic sorbent and metal catalyst component are integrated into a single material.

Aspect 2 provides the material of Aspect 1, wherein the solid inorganic sorbent comprises a carbonate.

Aspect 3 provides the material of any of Aspects 1 or 2, wherein the metal catalyst component comprises iron, cobalt, copper, manganese or any combination of oxides thereof.

Aspect 4 provides the material of any of Aspects 1-3, wherein the solid inorganic sorbent comprises group 1 metal carbonates, wherein the solid inorganic sorbent comprises a group 1 metal carbonate comprising K₂CO₃, Na₂CO₃, Li₂CO₃, or a mixture thereof, supported on porous materials comprising Al₂O₃, Carbon, SiO₂ ZrO₂, aluminosilicate zeolites, or a mixture thereof.

Aspect 5 provides the material of any of Aspects 1-4, wherein metal catalyst component comprises iron.

Aspect 6 provides the material of any of Aspects 1-5, wherein the material is Fe/K₂CO₃/Al₂O₃.

Aspect 7 provides the material of any of Aspects 1-6, wherein the material has a CO₂ capture capacity in a range of from about 1.5 wt % to about 25 wt % at temperatures in a range of from about ambient temperatures to about 150° C. in the presence of water vapor.

Aspect 8 provides the material of any of Aspects 1-7, wherein the material is capable of converting captured CO₂ to (C₁-C₁₀)hydrocarbyl products upon hydrogenation.

Aspect 9 provides the material of Aspect 8, wherein the (C₁-C₁₀) hydrocarbyl products comprise olefins, paraffins, or both.

Aspect 10 provides the material of any of Aspects 1-9, wherein the material comprises Fe particles impregnated on K₂CO₃/Al₂O₃.

Aspect 11 provides the material of Aspect 10, wherein the Fe particles range from about 2 wt % to about 50 wt % of the material.

Aspect 12 provides the material of any of Aspects 10 or 11, wherein the Fe particles range from about 20 wt % to about 30 wt % of the material.

Aspect 13 provides the material of any of Aspects 1-12, wherein the material is capable of capturing CO₂ from air containing 350 ppm CO₂ to about 1000 ppm CO₂.

Aspect 14 provides the material of any of Aspects 1-12, wherein the material is capable of capturing CO₂ from concentrated sources containing about 1 vol % CO₂ to about 30 vol % CO₂.

Aspect 15 provides the material of any of Aspects 1-14, wherein the material is capable of converting captured CO₂ to (C₁-C₁₀)hydrocarbyls with a selectivity of at least 10% for C₂-C₄ olefins or paraffins.

Aspect 16 provides the material of any of Aspects 1-15, wherein the material is capable of converting captured CO₂ to (C₁-C₁₀)hydrocarbyls at temperatures in a range of from about 250° C. to about 400° C.

Aspect 17 provides the material of any of Aspects 1-16, wherein the material is capable of converting captured CO₂ to (C₁-C₁₀)hydrocarbyls at temperatures in a range of from about 330° C. to about 360° C.

Aspect 18 provides a method for integrated direct air capture and catalytic conversion of CO₂ to (C₁-C₁₀)hydrocarbyl products, comprising:
contacting a multifunctional material comprising a solid inorganic sorbent and a metal catalyst component with dilute CO₂ gas streams to capture CO₂; and
hydrogenating the captured CO₂ to produce (C₁-C₁₀)hydrocarbyls.

Aspect 19 provides the method of Aspect 18, wherein the solid inorganic sorbent comprises a carbonate.

Aspect 20 provides the method of any of Aspects 18 or 19, wherein the solid inorganic sorbent comprises a group 1 metal carbonate comprising K₂CO₃, Na₂CO₃, Li₂CO₃, or a mixture thereof, supported on porous materials comprising Al₂O₃, Carbon, SiO₂ ZrO₂, aluminosilicate zeolites, or a mixture thereof.

Aspect 21 provides the method of any of Aspects 18-20, wherein the metal catalyst component comprises iron, cobalt, copper, manganese or any combination of oxides thereof.

Aspect 22 provides the method of any of Aspects 18-21, wherein the multifunctional material is Fe/K₂CO₃/Al₂O₃.

Aspect 23 provides the method of any of Aspects 18-22, wherein the CO₂ capture is performed at temperatures in a range of from about ambient temperatures to about 150° C. in the presence of water vapor.

Aspect 24 provides the method of any of Aspects 18-23, wherein the hydrogenation occurs at temperatures in a range of from about 300° C. to about 400° C.

Aspect 25 provides the method of any of Aspects 18-24, wherein the hydrogenation occurs at temperatures in a range of from about 332° C. to about 360° C.

Aspect 26 provides the method of any of Aspects 18-25, wherein the (C₁-C₁₀)hydrocarbyl products comprise olefins, paraffins, or both.

Aspect 27 provides the method of any of Aspects 18-26, further comprising recycling the multifunctional material for multiple cycles of CO₂ capture and conversion.

Aspect 28 provides the method of any of Aspects 18-27, wherein the dilute CO₂ gas stream is air, which contains about 350 ppm CO₂ to about 1000 ppm CO₂.

Aspect 29 provides the method of any of Aspects 18-28, wherein the dilute CO₂ stream gas is an industrial flue gas source containing about 1 vol % CO₂ to about 30 vol % CO₂.

Aspect 30 provides the method of any of Aspects 18-29, wherein the hydrogenation is performed under a hydrogen pressure in a range of from about 0.5 MPa to about 3 MPa.

Aspect 31 provides the method of any of Aspects 18-30, wherein the hydrogenation is performed under a hydrogen pressure in a range of from about 0.8 MPa to about 1.2 MPa.

Aspect 32 provides the method of any of Aspects 18-31, wherein the method achieves a selectivity in a range of from about 10% to about 90% for C₂-C₄ olefins.

Aspect 33 provides the method of any of Aspects 18-32, wherein the method converts at least 20% of the captured CO₂ to C2+ products.

Aspect 34 provides the method of any of Aspects 18-33, further comprising pretreating the multifunctional material with H₂ at a temperature in a range of about 250° C. to about 450° C. prior to CO₂ capture.

Aspect 35 provides the method of any of Aspects 18-34, further comprising pretreating the multifunctional material with an H₂/CO gas mixture.

Aspect 36 provides the method of any of Aspects 18-35, further comprising separating the produced (C₁-C₁₀)hydrocarbyls using a demethanization tower operated at cryogenic conditions.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. A comma can be used as a delimiter or digit group separator to the left or right of a decimal mark; for example, “0.000,1” is equivalent to “0.0001.” All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain aspects there is no hydrocarbyl group. A hydrocarbylene group is a diradical hydrocarbon, e.g., a hydrocarbon that is bonded at two locations

What is claimed is:

1. A multifunctional material for integrated capture and catalytic conversion of CO₂, comprising:
a solid inorganic sorbent; and
a metal catalyst component,
wherein the solid inorganic sorbent and metal catalyst component are integrated into a single material.

2. The material of claim 1, wherein the solid inorganic sorbent comprises a carbonate.

3. The material of claim 1, wherein the metal catalyst component comprises iron, cobalt, copper, manganese or any combination of oxides thereof.

4. The material of claim 1, wherein the solid inorganic sorbent comprises group 1 metal carbonates, wherein the solid inorganic sorbent comprises a group 1 metal carbonate comprising K₂CO₃, Na₂CO₃, Li₂CO₃, or a mixture thereof, supported on porous materials comprising Al₂O₃, Carbon, SiO₂ ZrO₂, aluminosilicate zeolites, or a mixture thereof.

5. The material of claim 1, wherein metal catalyst component comprises iron.

6. The material of claim 1, wherein the material is Fe/K₂CO₃/Al₂O₃.

7. The material of claim 1, wherein the material has a CO₂ capture capacity in a range of from about 1.5 wt % to about 25 wt % at temperatures in a range of from about ambient temperatures to about 150° C. in the presence of water vapor.

8. The material of claim 1, wherein the material is capable of converting captured CO₂ to (C₁-C₁₀)hydrocarbyl products upon hydrogenation.

9. The material of claim 8, wherein the (C₁-C₁₀) hydrocarbyl products comprise olefins, paraffins, or both.

10. The material of claim 1, wherein the material comprises Fe particles range from about 2 wt % to about 50 wt % of the material.

11. The material of claim 1, wherein the material is capable of capturing CO₂ from air containing 350 ppm CO₂ to about 1000 ppm CO₂, as well as from concentrated sources containing about 1 vol % CO₂ to about 30 vol % CO₂.

12. The material of claim 1, wherein the material is capable of converting captured CO₂ to (C₁-C₁₀)hydrocarbyls at temperatures in a range of from about 250° C. to about 400° C.

13. A method for integrated direct air capture and catalytic conversion of CO₂ to (C₁-C₁₀)hydrocarbyl products, comprising:
contacting a multifunctional material comprising a solid inorganic sorbent and a metal catalyst component with air to capture CO₂; and
hydrogenating the captured CO₂ to produce (C₁-C₁₀)hydrocarbyls.

14. The method of claim 13, wherein the solid inorganic sorbent comprises a carbonate.

15. The method of claim 13, wherein the metal catalyst component comprises iron, cobalt, copper, manganese or any combination of oxides thereof.

16. The method of claim 13, wherein the multifunctional material is Fe/K₂CO₃/Al₂O₃.

17. The method of claim 13, wherein the hydrogenation occurs at temperatures in a range of from about 300° C. to about 400° C.

18. The method of claim 13, wherein the (C₁-C₁₀)hydrocarbyl products comprise olefins, paraffins, or both.

19. The method of claim 13, wherein the air contains about 350 ppm CO₂ to about 1000 ppm CO₂.

20. The method of claim 13, wherein the hydrogenation is performed under a hydrogen pressure in a range of from about 0.5 MPa to about 3 MPa.
